(19) United States
(12) Patent Application Publication
(10) Pub. No.: US 2022/0062548 A1
(43) Pub. Date: Mar. 3, 2022

(54) POST MEAL COMPENSATION FOR AUTOMATIC INSULIN DELIVERY SYSTEMS

(71) Applicant: Insulet Corporation, Acton, MA (US)

(72) Inventors: Yibin ZHENG, Hartland, WI (US); Rangarajan NARAYANASWAMI, Weston, MA (US); Joon Bok LEE, Acton, MA (US)

(21) Appl. No.: 17/406,209

(22) Filed: Aug. 19, 2021

Related U.S. Application Data
(60) Provisional application No. 63/072,485, filed on Aug. 31, 2020.

(51) Int. Cl. A61M 5/172; G16H 40/67; G16H 20/17; G16H 40/40; G16H 10/60; G16H 50/70; G01N 33/49

(52) U.S. Cl. CPC A61M 5/1723; G16H 20/60; G16H 20/17; G16H 40/40; G16H 10/60; G16H 50/70; G01N 33/49; A61M 2205/52; A61M 2205/505; A61M 2205/50; A61M 2202/07; A61M 2205/3569; A61M 2230/201; A61M 2205/80; G16H 40/67

(57) ABSTRACT

Disclosed are systems, devices, methods and computer-readable medium products that compensate for consumption of a meal by providing a reduced-constraint meal bolus dosage. The reduced-constraint meal bolus dosage is determined using post-prandial safety constraints that are reduced based on an indication that a meal has been consumed. The post-prandial safety constraints are intended to protect users from overdelivering or underdelivering insulin to the user. The determinations may be performed by a control algorithm-based drug delivery system that enables automatic delivery of a drug, such as insulin or the like.

### CROSS-REFERENCE TO RELATED APPLICATIONS

This application claims the benefit to U.S. Provisional Application No. 63/072,485, filed Aug. 31, 2020, the entire contents of which are incorporated herein by reference in their entirety.

### TECHNICAL FIELD

The described examples provide a device, a computer readable medium and techniques for an automatic insulin delivery (AID) system to provide a more aggressive and rapid response to ingestion of a meal.

### BACKGROUND

Meals are among the most difficult challenges in glucose control for people with Type-1 diabetes mellitus. Prior automatic delivery systems may require the user to identify specific elements of a meal being ingested by the user, such as identifying when a meal is consumed, the composition of the meal (e.g., meat, starch, fruit, and the like), the estimated carbohydrates and/or calories in the meal, or the other user provided inputs.

Although compensation for meals is typically addressed through manual delivery of a bolus of insulin, there are many difficulties that limit the effectiveness of such treatments. These include: 1) difficulty in assessing the size of the meals, 2) difficulty in assessing the absorption rate of the meals, 3) difficulty in assessing the timing of the meals (i.e., time of insulin delivery versus time of meal ingestion), and 4) difficulty in assessing the correct IC ratio to calculate the proper insulin amount for the size of the meals. Further, requiring a manual calculation for every meal, every day for the rest of the lives of people with Type-1 diabetes mellitus results in substantial reduction in the quality of life of these patients.

These burdens on the patients that are a nuisance to the patient also delay the reduction of the user's blood glucose measurement values because the AID system reacts to the consumed meal in a more conservative manner.

### SUMMARY

Disclosed is a device including a processor, a memory, a touchscreen, and a transceiver. The memory may store programming code, an artificial pancreas application, and be operable to store data related to the artificial pancreas application. The programming code and the artificial pancreas application are executable by the processor. The touchscreen is operable to present a graphical user interface and receive inputs. The transceiver is operable to receive and transmit signals containing information usable by or generated by the artificial pancreas application. The artificial pancreas application includes post-prandial safety constraints related to administering of insulin in response to consumption of a meal. The processor when executing the artificial pancreas application is operable to receive an indication of consumption of a meal. The processor, in response to the indication of consumption of the meal, may decrease the post-prandial safety constraints from initial settings to a first level of reduced safety constraints. A reduced-constraint meal bolus dosage may be calculated using the first level of reduced safety constraints. A signal may be generated to actuate delivery of insulin according to the calculated reduced-constraint meal bolus dosage.

Disclosed is an example of a non-transitory computer readable medium that is embodied with programming code executable by a processor. the processor when executing the programming code is operable to maintain track of an amount of pre-existing insulin-on-board for a user. An announcement of a meal that is a signal in response to an indication of meal-related activity by the user may be received. A reduced-constraint meal bolus dosage may be determined based on a target blood glucose value, the amount of pre-existing insulin-on-board for the user and revised safety constraints. An amount of time that has elapsed since a last bolus was delivered may be determined. The amount of elapsed time may be compared to a threshold. In response to a result of the comparison indicating the elapsed time is less than the threshold, a signal may be generated to actuate a pump mechanism to deliver the meal bolus dosage.

Disclosed is a system that includes a personal diabetes management device and a drug delivery device. The personal diabetes management device may include a processor, a memory, a touchscreen and a transceiver. The memory stores programming code, an artificial pancreas application, and post-prandial safety constraints, and data related to the artificial pancreas application. The programming code and the artificial pancreas application are executable by the processor. The touchscreen is operable to present a graphical user interface; and the transceiver is operable to receive and transmit signals containing information of the artificial pancreas application. The drug delivery device includes a pump mechanism and a reservoir. The pump mechanism may be operable to be removably coupled to a user. The reservoir may be operable to contain insulin and is fluidly coupled to the pump mechanism. The processor of the personal diabetes management device when executing the artificial pancreas application is operable to receive an indication of consumption of a meal. In response to the indication of consumption of the meal, the post-prandial safety constraints may be decreased from initial settings to a first level of reduced safety constraints. The processor of the personal diabetes management device may calculate a reduced-constraint meal bolus dosage using the first level of reduced safety constraints; and generate an actuation signal to actuate delivery of insulin according to the calculated reduced-constraint meal bolus dosage. The pump mechanism may be communicatively coupled to the processor of the personal diabetes management device. The pump mechanism may be operable to expel, in response to the actuation signal from the processor, insulin from the reservoir according to the calculated reduced-constraint meal bolus dosage.

Disclosed is a method that includes receiving an indication of consumption of a meal. An amount of insulin to be delivered as a partial reduced-constraint meal bolus dosage may be determined using meal bolus safety constraints that are reduced to a first level based on the received indication. Delivery of the partial reduced-constraint meal bolus dosage may be actuated. Another indication confirming consumption of the meal may be received. A follow-up meal bolus dosage may be determined based on meal bolus safety constraints reduced to a second level from the first level. Delivery of insulin may be actuated according to the follow-up meal bolus dosage.

### BRIEF DESCRIPTION OF THE DRAWINGS

FIG. 1 shows a flow chart of an example process for determining a dosage of a bolus injection for correcting a blood glucose level.

FIG. 2 illustrates a functional block diagram of drug delivery system suitable for implementing the example processes and techniques described herein.

FIG. 3 illustrates a flow chart of another example process for delivering meal bolus dosages to compensate for consumption of a meal.

FIG. 4 illustrates an example of a user interface usable with the examples described in FIGS. 1-3.

FIG. 5 is a graphic illustrating analysis of blood glucose measurement values received from a blood glucose sensor according to examples described herein.

FIG. 6 is a flow chart of an example of a process for generating and using a machine-learning model according to some aspects of the disclosed examples.

### DETAILED DESCRIPTION

Various examples provide a method, a system, a device and a computer-readable medium for responding to the ingestion of a meal by an automatic insulin delivery system.

The disclosed examples provide techniques that may be used with any additional algorithms or computer applications that manage blood glucose levels and insulin therapy to provide a control algorithm-based drug delivery system. Such control algorithms may collectively be referred to as an “artificial pancreas” algorithm-based system, or more generally, an artificial pancreas (AP) application, that provides automatic delivery of insulin based on a blood glucose sensor input, such as that received from a CGM or the like. In an example, the artificial pancreas (AP) application when executed by a processor may enable a system to monitor a user's glucose values, determine an appropriate level of insulin for the user based on the monitored glucose values (e.g., blood glucose concentrations or blood glucose measurement values) and other information, such as user-provided information that may include, for example, carbohydrate intake, exercise times, meal times or the like, and take actions to maintain a user's blood glucose value within an appropriate range. A target blood glucose value setting of the particular user may alternatively be a range of blood glucose measurement values that are appropriate for the particular user. For example, a target blood glucose measurement value may be acceptable if it falls within the target blood glucose value setting range of 80 mg/dL to 120 mg/dL, which is a range satisfying the clinical standard of care for treatment of diabetes. However, an AP application as described herein may be able to establish a target blood glucose value setting more precisely and may set the target blood glucose value setting at, for example, 110 mg/dL, or the like.

The term “target blood glucose value setting” may refer to settings within the AP application that may be settings established by operation of the AP application over time based on user information (such as blood glucose measurement value history), settings provided as an input into the AP application by a user, a user's guardian, a physician, a clinician, or the like, or be default settings set by the AP application. A “blood glucose measurement value” may refer to the results of blood glucose measurements made by a blood glucose sensor, such as a continuous glucose monitor, a blood glucose monitor that is operated by a user, or the like.

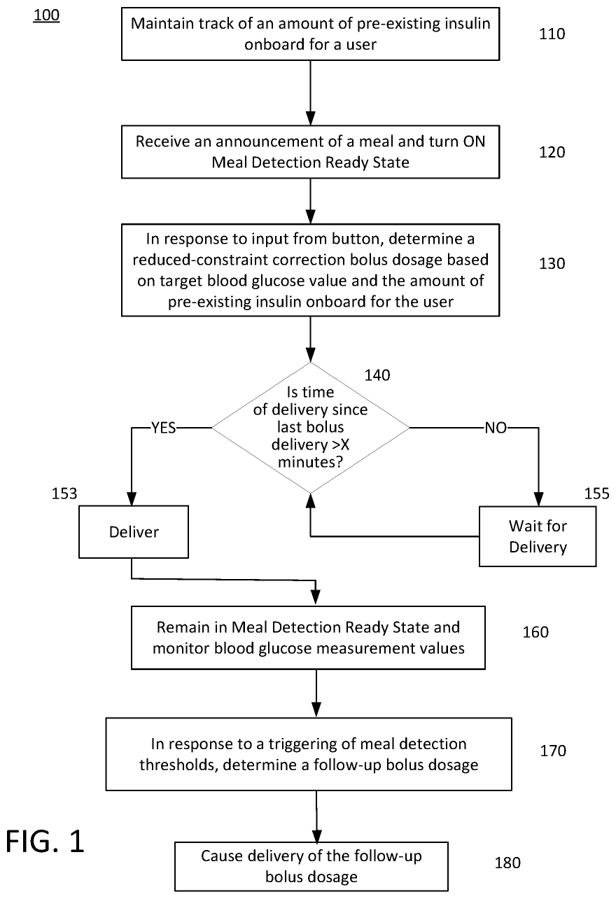

As described in more detail with reference to the examples of FIGS. 1-6, the AP application may monitor a user's blood glucose measurement values, inputs from a user interface or a meal detection and response algorithm and may utilize the monitored information and/or the inputs to reduce safety constraints that may be used in the determination of a meal correction bolus dosage. The inputs from the user interface or the meal detection and response algorithm may be indications announcing that the user consumed or is about to consume a meal. The meal correction bolus dosage is intended to compensate for the increase in blood glucose that results from the carbohydrates in the consumed meal. The devices and system may also be operable to generate and send a command to a medical device, that includes a pump mechanism, to control delivery of a meal correction bolus dose of insulin to the user as well as to control other functions.

When responding to a meal, algorithms of the AP application without aid of the functions illustrated in the following examples implement a conservative approach due to uncertainty of the actual ingestion of a meal built into the respective meal detection algorithm. In contrast to this conservative approach, the disclosed examples may implement an aggressive delivery of insulin to more quickly, yet appropriately, compensate for consumption of a meal that adheres to reduced or decreased safety constraints. The following examples provide an AP application that is configured with a meal detection and response algorithm that is operable to modify post-prandial safety constraints that permit delivery of an amount of insulin to be administered to a user that more quickly compensates for consumption of the meal. As explained in more detail below, the examples of a meal detection and response algorithm may indicate the ingestion of a meal that enables the AP application to modify safety constraint settings for determination of the meal bolus, which enables more rapid compensation for meals. For instance, a default safety constraint setting may limit the range of the total amount of insulin that is delivered to compensate for a meal ingestion from 0% to an amount below the full amount needed, such as 80%. This is due to the need for the system to modulate its insulin delivery in case the user's meal ingestion estimate is not accurate. In contrast, the AP application utilizing a meal detection and response application examples as discussed herein may relax safety constraints to modify the AID algorithm's compensation for meal ingestion to permit delivery of the full 100% of the amount needed to modulate this insulin delivery. For example, the AP application may relax the default safety constraints that would be active during cases without a meal. In an example, the safety constraints that may be relaxed include the maximum delivery limit per control cycle of the system (such as 5 minutes or the like), total insulin delivery limit over a defined period (such as no more than a fixed amount over number of hours of system use, e.g., 3 hours or the like), or the constraints based on recent insulin deliveries (insulin-on-board constraints).

An advantage of the disclosed examples is an automatic insulin delivery (AID) system enabled to determine that a meal has been consumed and modify safety constraints related to the consumed meal to enable the automatic insulin delivery system to quickly and seamlessly administer an appropriate amount of insulin without requiring a user to input details related to the consumed meal. Details related to the consumed meal may include identification of the composition of the meal (e.g., meat, starch, fruit, and the like), estimated number of carbohydrates and/or calories in the meal, meal size, estimated number of calories or carbohydrates, or the like. Using the described techniques, the system reduces the burden on the user when it is time to deliver insulin to compensate for changes in blood glucose measurement values as a result of consuming a meal and optimizes delivery of a meal correction bolus so a user may more quickly receive their bolus and begin lowering their blood glucose measurement value.

It may be helpful to describe in more detail the above examples as well as other examples of reducing safety constraints related to determining meal correction bolus dosages with reference to the drawings.

The AP application controlling the AID system may have default safety constraint settings to account for consumption of a meal. Often the default safety constraint settings are conservative with respect to the amounts of insulin to be delivered to the user and how quickly excursions of the user's blood glucose measurement value away from the user's target blood glucose value setting are addressed. By adhering to the conservative default safety constraints, the AP application will, after a time, return the user's blood glucose measurement value to within range of the user's target blood glucose measurement value.

FIG. 1 shows a flow chart of a process for determining a dosage of a bolus injection for correcting a blood glucose level in response to a user consuming a meal. During operation of the AP application outside of when a meal is consumed, the AID system may be designed to determine if a blood glucose measurement value is above a target blood glucose value setting and deliver insulin to bring the blood glucose measurement value back to within a range (e.g., within 0-2% of target blood glucose value setting) of the target blood glucose value setting. Recall that prior systems may administer a single recommended correction bolus to compensate for a meal and the single recommended correction bolus may have an amount of insulin to account for the entirety of the meal.

The process 100 may be implemented by programming code that is executed by a processor and is intended to return the user's blood glucose measurement value to within range of the user's target blood glucose value setting without relying on the default approach that utilizes the conservative safety constraints. The processor, when executing the programming code may be configured to maintain track of an amount of pre-existing insulin-on-board for a user (110). Insulin-on-board for a user may be defined as an amount of insulin that the user's body has not yet processed and that will be effective in lowering a user's blood glucose measurement value. The programming code may implement a function of an AP application that assists with controlling an AID system. The AP application may be operable to receive the date, the time and the amounts of insulin delivered by the AID system to the user over the course of hours, days, weeks and months, which may be stored in a memory of a personal diabetes monitoring device or the like.

In addition, the AP application may be operable to receive blood glucose measurement values with or without date and time information of each respective blood glucose measurement value from a user or a continuous glucose monitoring system or similar sensor. The AP application may also receive only a blood glucose measurement value from the user, the continuous glucose monitoring system or the similar sensor, and may provide date and time information. For example, the AP application may be operable to assign a date and time stamp when a blood glucose measurement value is received. The AP application may use the received blood glucose measurement values with the respective date and time information. For example, based on the blood glucose measurement values, the amount of insulin may be delivered, and other information related to a user's physiology (e.g., insulin sensitivity and the like), the AP application may be operable to determine the user's insulin-on-board (JOB).

The process 100 may proceed with the AP application receiving an announcement of a meal and the meal detection ready state may be turned ON (120). The meal announcement may be a signal in response to an indication of meal-related activity by the user. The meal announcement may be received in a various ways by the AP application. The AP application may receive a “meal announcement,” which in one example, may be an indication of a user interaction with a meal announcement button on a graphical user interface. In another example, the meal announcement may be generated in response to an indication from the meal detection and response algorithm that determined that a user consumed a meal. In a further example, the AP application may, in response to the meal announcement, turn ON a “meal detection ready state”, which modifies settings of the meal detection and response application to enable a more rapid response to indicators of meal consumption, such as increasing blood glucose measurement values.

An example of how the indication of a meal and the meal announcement are generated may be based on the processor receiving a number of blood glucose measurement values over a period of time. The period of time may include time before receipt of the meal announcement and time after receipt of the meal announcement. Alternatively, the period of time may only include time before receipt of the meal announcement. In yet a further alternative, the period of time may only include time after receipt of the meal announcement. A meal detection and response algorithm that is part of the programming code of the AP application or coupled to the AP application as a plug-in to the AP application may be executed by the processor and may analyze the number of blood glucose measurement values over the period of time. Using the received number of blood glucose measurement values, the meal detection and response application may be operable to generate an indication of the meal announcement.

The AP application of an AID system determines a meal bolus based on what a user's body needs as determined by analysis of their blood glucose measurement values. The AP application may be operable to determine a reduced-constraint meal bolus dosage based on a target blood glucose value, the amount of pre-existing insulin-on-board for the user, and revised safety constraints (130).

For example, if a meal indication has been received and the blood glucose measurement values continue to rise, the system can be confident that a meal has been ingested. For example, if the total area under the curve between the user's glucose and the blood glucose measurement values accumulate to more than 750-3000 (mg/dL)*minute, the system may deliver a meal bolus equivalent to compensate for the user's current glucose without accounting for prior JOB. The system may allow this to be repeated no more than once every, for example, 15-60 minutes, and no more than 3-9 times in total. In a specific example, the blood glucose measurement values accumulate to 1500 (mg/dL)/minute, or more than 50 mg/dL above the target blood glucose value setting (i.e., 110 mg/dL) for more than 30 minutes. The AP application may be set to interpret the 30-minute duration for exceeding the target blood glucose value setting as being a consistent time above the target that requires triggering of a meal detection indication. For example, the user's blood glucose measurement values may have consistently been above the user's target blood glucose value setting for a period of time, such as for example, 110-150 mg/DL for 1 hour. When this information is combined with the indication from the interaction with the meal announcement button from 110, the process 100 may deliver insulin that meets the need for the correction of the user's blood glucose measurement value back to the user's target blood glucose measurement value.

In the specific example, the time period before a bolus may be repeated may be 30 minutes, with a setting that a delivery of a meal bolus may not occur more than 3 times in total. This limit of 3 times is based on being conservative with respect to a meal. For example, a user may drink a small sugary soda that is causing the rise in blood glucose measurement values, but the soda is too small to sustain this increase, so in order to avoid overcorrecting, the reduced-constraint meal bolus may only be a partial bolus.

In a further example, the determination at step 130 may be based on heuristics and a user's meal bolus dosage may be determined according to a reduction in a user's safety constraints. Safety constraints are settings in the AP application and algorithms that limit the amount of insulin that may be delivered to the user and timing of the delivery of the respective amounts of insulin. Different safety constraints may be set for different situations and/or time-of-day. For example, safety constraints of the AP application for meal boluses may differ from safety constraints for the basal delivery of insulin that occurs throughout the day. In addition, the safety constraints may be tailored for each individual over time. For example, when a user initially begins use of an AID system, the safety constraints in the AP application may be set to default settings. The AP application may modify the default settings based on the user's physiology gleaned from analysis of the user's blood glucose measurement values, inputs from the user to indicate user preferences, and other factors to provide safety constraints tailored to the user and their lifestyle.

For example, the processor when executing the programming code to determine the reduced-constraint meal bolus dosage may be operable to obtain an amount of insulin-on-board for the user. In the example, the amount of insulin-on-board may be an estimate of the amount of insulin that remains effective for reducing the user's blood glucose. The recommended dosage of insulin (also referred to as a recommended meal bolus dosage) may be modified by subtracting the obtained amount of insulin-on-board from the recommended dosage; and the system may output the modified recommended dosage of insulin as the reduced-constraint meal bolus dosage.

In another example of determining the reduced-constraint meal bolus dosage, the processor, when executing the programming code to determine the reduced-constraint meal bolus dosage, may be operable to obtain a total daily insulin of the user. The processor may be further operable to obtain a user's blood glucose measurement values over a period of time and use the obtained user's total daily insulin and the obtained blood glucose measurement values in the determination of the reduced-constraint meal bolus dosage.

In a further example, the processor, when executing the programming code to determine the reduced-constraint meal bolus dosage, may be operable to determine a difference between the blood glucose measurement value of the user at a time when the meal announcement was received and the user's target blood glucose value. The processor may divide the difference by a correction factor related to the user's insulin sensitivity to provide a proposed meal bolus dosage. The correction factor may be specific to the user based on the user's insulin sensitivity. The insulin sensitivity may be a measure of how efficiently the user's body processes the administered insulin. To be more precise, the insulin sensitivity may be a measure how much blood glucose concentration is lowered by a unit of administered insulin for the user. The processor may further determine an amount of insulin-on-board for the user. Using the determined amount of insulin-on-board for the user, the processor may determine the reduced-constraint meal bolus dosage by subtracting the determined amount of insulin-on-board for the user from the proposed meal bolus dosage.

At 140, the processor may determine an amount of time that has elapsed since a last bolus was delivered and compare the amount of elapsed time to a threshold, such as X minute, where X may be 45, 60, 90 or the like. In response to a result of the comparison indicating the elapsed time is greater than the threshold, the processor may generate a signal to actuate a pump mechanism to deliver the meal bolus dosage (153). In a second part that is in response to an indication from the meal announcement button, the AID system may deliver a fixed amount of insulin (e.g., 10 Units of insulin, 25 Units of insulin, or the like) and enter a meal detection ready state.

Conversely, in response to a result of the comparison indicating the elapsed time is less than the threshold, the processor may wait for delivery and return to 140 to monitor the elapsed time until it is greater than the threshold (155).

The AP application may maintain the meal detection and ready state and monitor blood glucose measurement values (160) for trends as the blood glucose measurement values are received from a BG sensor. The AP application may implement a meal detection and response algorithm that remains in the meal detection ready state for a period of time, such as, for example, 60-120 minutes. In response to detection of a meal while in the meal detection ready state, there are now two confirmations of a detected meal: a user action (interaction with the meal announcement button) and a characteristic change in blood glucose measurement value that indicates consumption of a meal by the user. The characteristic change in blood glucose measurement value may be an upward trend of blood glucose measurement values. This allows the system to be more confident that additional insulin delivery is still safe for the user.

In response to a triggering of meal detection thresholds, the system may determine a follow-up bolus dosage (170). In the specific example, the AP application may receive an indication of consumption of a meal in response to a user interaction with a meal announcement button on a graphical user interface (as in step 120). At the time of the user interaction with the meal announcement button, the AP application may determine a reduced-constraint meal bolus equivalent to 25-100% of the recommended correction bolus required to bring the user to a target blood glucose measurement value. For example, the target blood glucose value setting may be a range of 80-110 mg/dL, and the AP application may determine the amount of insulin for the recommended correction bolus. But to determine the partial reduced-constraint meal bolus dosage, the system may subtract any pre-existing insulin-on-board from the amount of insulin. Then the partial reduced-constraint meal bolus dosage may be administered to the user. This bolus delivery may only be performed, for example, no more than once every 30-90 minutes.

In a specific example, the partial reduced-constraint meal bolus dosage may, after subtracting any pre-existing insulin-on-board, be 50% of the recommended correction bolus that is calculated to return the user's target blood glucose value setting to 80 mg/dL. The reduced-constraint meal bolus dosage will also be delivered 90 minutes after any previous meal bolus. Continuing with the specific example, also in response to the received indication of consumption of the meal, the AP application may change settings within the meal detection and response algorithm. For example, the AP application may further reduce the sensitivity of the meal detection and response algorithm making it more probable that consumption of a meal is indicated (i.e., detection of a meal is more likely).

In a further example in which the meal detection and response algorithm has been informed that user is having a meal, meal detection has been triggered, and blood glucose measurement values are rising, the AP application may be operable to deliver another dosage of insulin as a follow-up meal bolus dosage to further compensate for the meal. To prevent over-compensating for the meal, the AP application may evaluate the rise in glucose to determine whether the rise is a consistent rise in glucose suggesting ingestion of a meal. For example, a consistent rise of 3-4 (mg/dL)/minute or even 2 (mg/dL)/minute for 15-30 minutes or 20-30 minutes may be considered by the meal detection and response algorithm as a glucose rise rate that is consistent with ingestion of a meal.

At 180, the AP application cause delivery of the follow-up bolus dosage by sending an actuation signal to the drug delivery device. The actuation signal may include commands operable to cause delivery of insulin as well as an amount of insulin to be delivered that is equal to the amount in the follow-up meal bolus dosage. The drug delivery device in response to the actuation signal may actuate a pump mechanism to deliver the follow-up dosage of insulin.

Alternatively, the AP application may be operable to respond to a meal even when a user does not interact with a meal announcement button. For example, at any point, if the user experiences glucose excursions similar to a meal, the AP application may be operable to deliver additional insulin periodically, such as providing a partial meal correction bolus several times during the day. The several times may be 3 times a day, 2 times a day, or within the range of 1-5 times a day based on as user's blood glucose measurement values during the day.

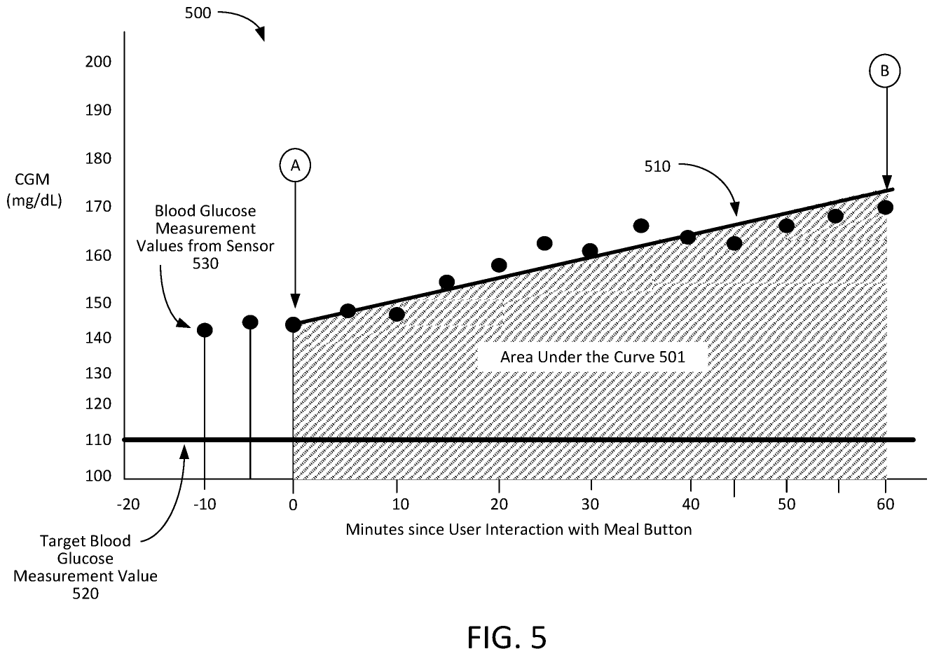

Another alternative example with respect to the blood glucose measurement value curve is described in more detail in FIG. 5 below. In general, however, if the total area under the curve between the user's blood glucose measurement value and target blood glucose value setting accumulates to more than 750-3000 (mg/dL*minute), the AP application may be operable to deliver, for example, 65%, 50%, 40%, or the like of a correction bolus equivalent to bring the user's blood glucose measurement value to within range (e.g., 110-140 mg/dL) of the user's target blood glucose value without accounting for prior IOB. The AP application may be operable to limit this alternative to be repeated, for example, only after 30 minutes since last administering the 50% or other % correction bolus and no more than 2-4 times per detected consumption of a meal.

In another alternative at 170, the AP application may implement a response that may be executed more rapidly based on the user's glucose excursion as discussed above. If the user's glucose is more than approximately 30-100 mg/dL above target blood glucose value setting and has a positive rate of change for more than approximately 15-30 minutes, then the system may repeat delivery of a correction bolus periodically without subtracting any previous IOB.

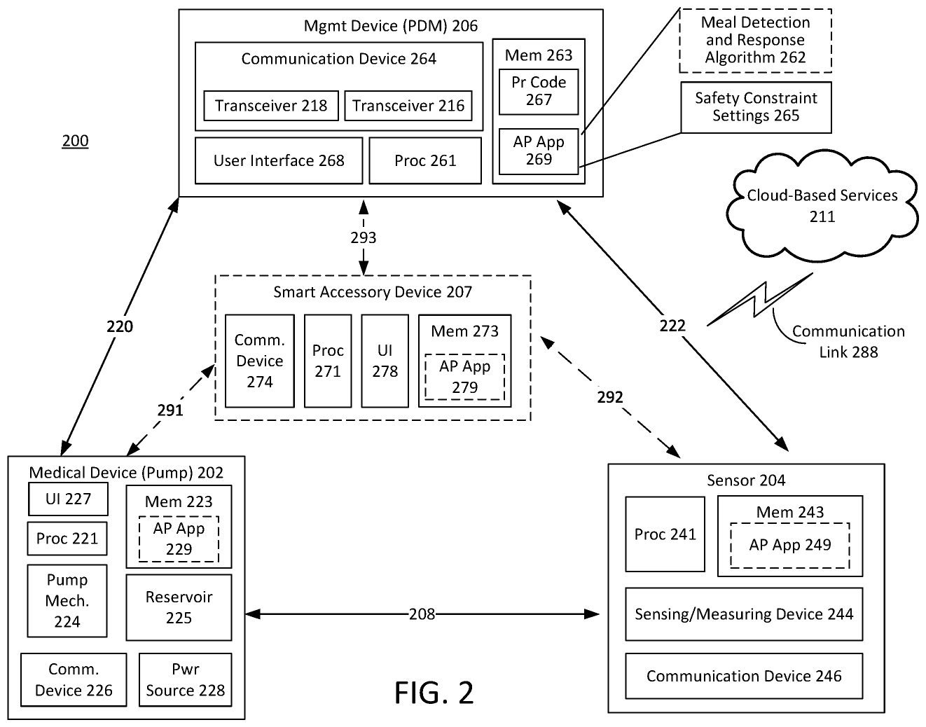

It may be helpful to discuss an example of a drug delivery system that may implement the process example of FIG. 1. FIG. 2 illustrates an example of a drug delivery system 200.

The drug delivery system 200 may be operable to implement an AP application that includes functionality to determine a bolus dosage, output an indication of the determined bolus dosage to actuate delivery of the bolus of insulin based on the indication of the determined bolus dosage. The drug delivery system 200 may be an automated drug delivery system that may include a medical device (pump) 202, a sensor 204, and a personal diabetes management device (PDM) 206. The system 200, in an example, may also include a smart accessory device 207, which may communicate with the other components of system 200 either via a wired or wireless communication link.

The medical device 202 may be attached to the body of a user, such as a patient or diabetic, and may deliver any therapeutic agent, including any drug or medicine, such as insulin or the like, to a user. The medical device 202 may, for example, be a wearable device worn by the user. For example, the medical device 202 may be directly coupled to a user (e.g., directly attached to a body part and/or skin of the user via an adhesive or the like). In an example, a surface of the medical device 202 may include an adhesive to facilitate attachment to a user.

The medical device 202 may include a number of components to facilitate automated delivery of a drug (also referred to as a therapeutic agent) to the user. The medical device 202 may be operable to store the drug and to provide the drug to the user. The medical device 202 is often referred to as a pump, or an insulin pump, in reference to the operation of expelling a drug from the reservoir 225 for delivery to the user. While the examples refer to the reservoir 225 storing insulin, the reservoir 225 may be operable to store other drugs or therapeutic agents, such as morphine or the like, suitable for automated delivery.

In various examples, the medical device 202 may be an automated, wearable insulin delivery device. For example, the medical device 202 may include a reservoir 225 for storing the drug (such as insulin), a needle or cannula (not shown) for delivering the drug into the body of the user (which may be done subcutaneously, intraperitoneally, or intravenously), and a pump mechanism (mech.) 224, or other drive mechanism, for transferring the drug from the reservoir 225, through a needle or cannula (not shown), and into the user. The pump mechanism 224 may be fluidly coupled to reservoir 225, and communicatively coupled to the processor 221. The medical device 202 may also include a power source 228, such as a battery, a piezoelectric device, or the like, for supplying electrical power to the pump mechanism 224 and/or other components (such as the processor 221, memory 223, and the communication device 226) of the medical device 202. Although not shown, an electrical power supply for supplying electrical power may similarly be included in each of the sensor 204, the smart accessory device 207 and the personal diabetes management device (PDM) 206.

The blood glucose sensor 204 may be a device communicatively coupled to the processor 261 or 221 and may be operable to measure a blood glucose value at a predetermined time interval, such as every 5 minutes, or the like. In an example configuration, multiple instances of the AP application may operate on the respective devices 206, 202 and 207. The blood glucose sensor 204 may provide a number of blood glucose measurement values to the respective instances of the AP application operating on the respective devices, if the system 200 is so configured.

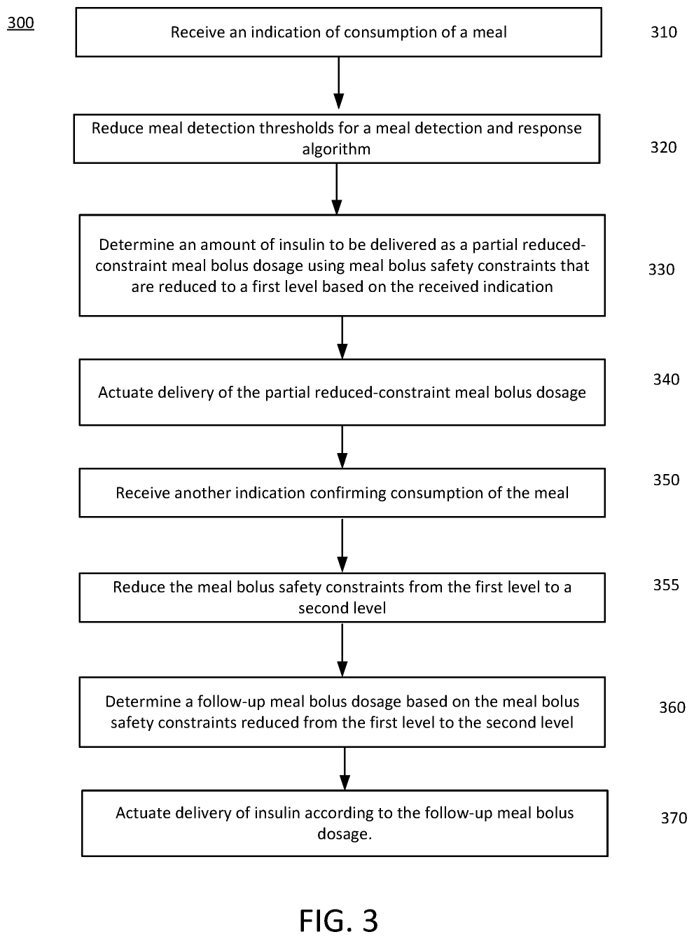

The medical device 202 may provide insulin the stored in reservoir 225 to the user based on information (e.g., blood glucose measurement values) provided by the sensor 204 and/or the management device (PDM) 206. For example, the medical device 202 may contain analog and/or digital circuitry that may be implemented as a processor 221 (or controller) for controlling the delivery of the drug or therapeutic agent. The circuitry used to implement the processor 221 may include discrete, specialized logic and/or components, an application-specific integrated circuit, a microcontroller or processor that executes software instructions, firmware, programming instructions or programming code (enabling, for example, the artificial pancreas application (AP App) 229 as well as the process examples of FIGS. 1 and 3) stored in memory 223, or any combination thereof. For example, the processor 221 may execute a control algorithm, such as an artificial pancreas application 229, and other programming code that may make the processor 221 operable to cause the pump to deliver doses of the drug or therapeutic agent to a user at predetermined intervals or as needed to bring blood glucose measurement values to a target blood glucose value. The size and/or timing of the doses may be programmed, for example, into an artificial pancreas application 229 by the user or by a third party (such as a health care provider, medical device manufacturer, or the like) using a wired or wireless link, such as 220, between the medical device 202 and a management device 206 or other device, such as a computing device at a healthcare provider facility. In an example, the pump or medical device 202 is communicatively coupled to the processor 261 of the management device via the wireless link 220 or via a wireless link, such as 291 from smart accessory device 207 or 208 from the sensor 204. The pump mechanism 224 of the medical device may be operable to receive an actuation signal from the processor 261, and in response to receiving the actuation signal, expel insulin from the reservoir 225 according to the set insulin bolus dosage.

The other devices in the system 200, such as management device 206, smart accessory device 207 and sensor 204, may also be operable to perform various functions including controlling the medical device 202. For example, the management device 206 may include a communication device 264, a processor 261, and a management device memory 263. The management device memory 263 may store an instance of the AP application 269 that includes programming code to implement functions as described herein as well as the meal detection and response algorithm 262 and safety constraint settings 265, that when executed by the processor 261 provides the process examples described with reference to the examples of FIGS. 1 and 3. The memory 263 may also store programming code to, for example, operate the user interface 268 (e.g., a touchscreen device, a camera or the like), communication device 264 and the like of the management device 206. The management device memory 263 may also store programming code for providing the process examples described with reference to the examples of FIGS. 1 and 3-7.

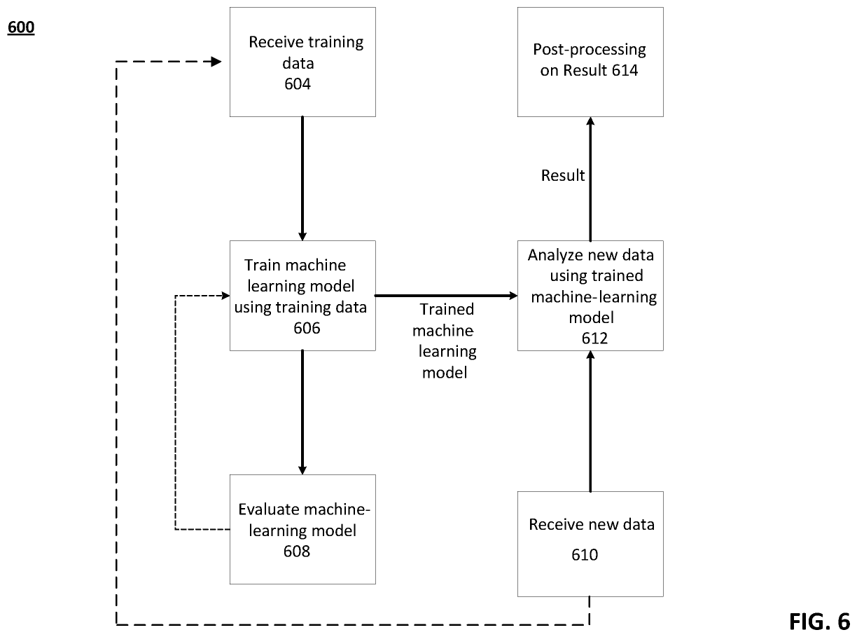

The meal detection and response algorithm, such as 262 of FIG. 2, may be configured to handle missed detections and to also mitigate against false positives. For example, the meal detection and response algorithm may utilize a machine-learning model (explained in more detail with reference to the example of FIG. 6) that may be trained on real blood glucose measurement data across multiple users with meal labels determined by defining glucose rise over time windows. In the examples, meal detection may be based on the blood glucose measurement record history in the recent past (up to two hours).

The smart accessory device 207 may be, for example, an Apple Watch®, other wearable smart device, including eyeglasses, provided by other manufacturers, a global positioning system-enabled wearable, a wearable fitness device, smart clothing, or the like. Similar to the management device 206, the smart accessory device 207 may also be operable to perform various functions including controlling the medical device 202. For example, the smart accessory device 207 may include a communication device 274, a processor 271, a user interface 278 and a memory 273. The user interface 278 may be a graphical user interface presented on a touchscreen display of the smart accessory device 207. The memory 273 may store an instance of the AP application 279 that includes programming code for providing the process examples described with reference to the examples of FIGS. 1 and 3-7. The memory 273 may also as store programming code and be operable to store data related to the AP application 279 and provide functionality for the respective components 271, 274 and 278 of smart accessory device.

Instructions for determining the delivery of the drug or therapeutic agent (e.g., as a bolus dosage) to the user (e.g., the size and/or timing of any doses of the drug or therapeutic agent) may originate locally by the management device 206 or may originate remotely (e.g., the cloud based services 211 or the smart accessory device 207) and be provided to the management device 206. In an example of a local determination of drug or therapeutic agent delivery, programming instructions, such as an instance of the artificial pancreas application 269, stored in the memory 263 that is coupled to the medical device 202 may be used to make determinations by the management device 206. In addition, the management device 206 may be operable to communicate with the cloud-based services 211 via the communication device 264 (via one or more of transceivers 216 or 218) and the communication link 288.

Instructions, such as command signals or actuation signals, may be provided to the medical device 202 over a wired or wireless link by the management device (PDM) 206, which has a processor 261 that executes an instance of the artificial pancreas application 269, or the smart accessory device 207, which has a processor 271 that executes an instance of the artificial pancreas application 269 as well as other programming code for controlling various devices, such as the medical device 202, smart accessory device 207 and/or sensor 204. The medical device 202 may execute any received instructions (originating internally or from the management device 206) for the delivery of the drug or therapeutic agent to the user. In this way, the delivery of the drug or therapeutic agent to a user may be automated.

In various examples, the medical device 202 may communicate via a wireless link 220 with the management device 206. The management device 206 may be an electronic device such as, for example, a smart phone, a tablet, a dedicated diabetes therapy management device, or the like. The management device 206 may be a wearable wireless accessory device. The wireless links 208, 220, 222, 291, 292 and 293 may be any type of wireless link provided by any known wireless standard. As an example, the wireless links 208, 220, 222, 291, 292 and 293 may enable communications between the medical device 202, the management device 206 and sensor 204 based on, for example, Bluetooth®, Wi-Fi®, a near-field communication standard, a cellular standard, or any other wireless optical or radio-frequency protocol.

The sensor 204 may be a glucose sensor operable to measure blood glucose at a predetermined time interval and output a blood glucose measurement value or data that is representative of a blood glucose value of a user. For example, the sensor 204 may be a glucose monitor or a continuous glucose monitor (CGM). Over time, the blood glucose sensor 204 provides the number of blood glucose measurement values to the personal diabetes management device 206. The sensor 204 may include a processor 241, a memory 243, a sensing/measuring device 244, and communication device 246. The memory 243 may store an instance of an AP application 249 as well as other programming code and be operable to store data related to the AP application 249. The AP application 249 may also include programming code for providing the process examples described with reference to the examples of FIGS. 1 and 3. The communication device 246 of sensor 204 may include one or more sensing elements, an electronic transmitter, receiver, and/or transceiver for communicating with the management device 206 over a wireless link 222 or with medical device 202 over the link 208. The sensing/measuring device 244 may include one or more sensing elements, such as a glucose measurement, heart rate monitor, pressure sensor, or the like. The processor 241 may include discrete, specialized logic and/or components, an application-specific integrated circuit, a microcontroller or processor that executes software instructions, firmware, programming instructions stored in memory (such as memory 243), or any combination thereof. For example, the memory 243 may store an instance of an AP application 249 that is executable by the processor 241.

Although the sensor 204 is depicted as separate from the medical device 202, in various examples, the sensor 204 and medical device 202 may be incorporated into the same unit. That is, in various examples, the sensor 204 may be a part of the medical device 202 and contained within the same housing of the medical device 202 (e.g., the sensor 204 may be positioned within or embedded within the medical device 202). Glucose monitoring data (e.g., measured blood glucose values) determined by the sensor 204 may be provided to the medical device 202, smart accessory device 207 and/or the management device 206 and may be used to determine a bolus dosage of insulin for automated delivery of insulin by the medical device 202.

In an example, the management device 206 may be a personal diabetes manager. The management device 206 may be used to program or adjust operation of the medical device 202 and/or the sensor 204. The management device 206 may be any portable electronic device including, for example, a dedicated controller device, a smartphone, or a tablet. In an example, the management device (PDM) 206 may include a processor 261, a management device management device memory 263, and a communication device 264. The management device 206 may contain analog and/or digital circuitry that may be implemented as a processor 261 (or controller) for executing processes to manage a user's blood glucose levels and for controlling the delivery of the drug or therapeutic agent to the user.

The processor 261 may also be operable to execute programming code stored in the management device management device memory 263. For example, the management device management device memory 263 may be operable to store an artificial pancreas application 269 that may be executed by the processor 261 The memory 263, in addition to storing the artificial pancreas application (AP app) 269 and data related to the AP application 269, may also store programming code 267 that when executed by the processor 261 implements functions that may provide supplemental information to the AP application 263. For example, the programming code 267 may implement a meal detection and response application and provide other functions, such as indications and notifications of a meal announcement and the like. The user interface 268 may, for example, be a touchscreen that under the control of the processor is operable to present a graphical user interface.

The communication device 264 may include one or more transceivers such as 216 and 218 and receivers or transmitters that operate according to one or more radio-frequency protocols. In the example, the respective transceivers 216 and 218 may be a cellular transceiver and a Bluetooth® transceiver. For example, the management device 206 may include a transceiver operable to receive and transmit signals containing information usable by the artificial pancreas application 269. Each transceiver 216 or 218 may be operable to receive signals. For example, each signal received from the sensor 204 may contain a respective blood glucose measurement value of a number of blood glucose measurement values. Alternatively, each signal from the sensor 204 may contain a batch of blood glucose measurement values with, for example, time stamps or the like. The respective transceivers of communication device 264 may be operable to transmit signals containing information useable by or generated by the AP application or the like. The communication devices 226, 244 and 276 of respective medical device 202, sensor 204 and smart accessory device 207 may also be operable to transmit signals containing information useable by or generated by the AP application or the like.

The medical device 202 may communicate with the sensor 204 over a wireless link 208 and may communicate with the management device 206 over a wireless link 220. The sensor 204 and the management device 206 may communicate over a wireless link 222. The smart accessory device 207, when present, may communicate with the medical device 202, the sensor 204 and the management device 206 over wireless links 291, 292 and 293, respectively. The wireless links 208, 220, 222, 291, 292 and 293 may be any type of wireless link operating using known wireless standards or proprietary standards. As an example, the wireless links 208, 220, 222, 291, 292 and 293 may provide communication links based on Bluetooth®, Wi-Fi, a near-field communication standard, a cellular standard, or any other wireless protocol via the respective communication devices 226, 246 and 264. In some examples, the medical device 202 and/or the management device 206 may include a user interface 227 and 268, respectively, such as a keypad, a touchscreen display, levers, buttons on a housing of the management device 206, a microphone, a camera, a speaker, a display, or the like, that is operable to allow a user to enter information and allow the management device 206 to output information for presentation to the user. The user interface 268 may provide inputs to programming code that interpret inputs received via the user interface 268, such as a voice input, a gesture (e.g., hand or facial) input to a camera, swipes to a touchscreen, or the like.

In various examples, the drug delivery system 200 may be an insulin drug delivery system. In various examples, the medical device 202 may be the OmniPod® (Insulet Corporation, Billerica, Mass.) insulin delivery device as described in U.S. Pat. Nos. 7,303,549, 7,137,964, or U.S. Pat. No. 6,740,059, each of which is incorporated herein by reference in its entirety.

In various examples, the drug delivery system 200 may implement the artificial pancreas (AP) algorithm (and/or provide AP functionality) to govern or control automated delivery of insulin to a user (e.g., to maintain euglycemia—a normal level of glucose in the blood). The AP application may be implemented by the medical device 202 and/or the sensor 204. The AP application may be used to determine the times and dosages of insulin delivery. In various examples, the AP application may determine the times and dosages for delivery based on information known about the user, such as the user's sex, age, weight, or height, and/or on information gathered about a physical attribute or condition of the user (e.g., from the sensor 204). For example, the AP application may determine an appropriate delivery of insulin based on blood glucose measurement values provided by the sensor 204. The AP application may also allow the user to adjust insulin delivery. In some examples, different functions of the AP application may be distributed among two or more of the management device 206, the medical device (pump) 202 or the sensor 204. In other examples, the different functions of the AP application may be performed by one device, such as the management device 206, the medical device (pump) 202 or the sensor 204.

As described herein, the drug delivery system 200 or any component thereof, such as the management device may be considered to provide AP functionality or to implement an AP application. Accordingly, references to the AP application (e.g., functionality, operations, or capabilities thereof) are made for convenience and may refer to and/or include operations and/or functionalities of the drug delivery system 200 or any constituent component thereof (e.g., the medical device 202 and/or the management device 206). The drug delivery system 200—for example, as an insulin delivery system implementing an AP application—may be considered to be a drug delivery system or an AP application-based delivery system that uses sensor inputs (e.g., data collected by the sensor 204).

In an example, one or more of the devices, 202, 204, 206 or 207 may be operable to communicate via a wireless communication link 288 with cloud-based services 211. The cloud-based services 211 may utilize servers and data storage (not shown). The communication link 288 may be a cellular link, a Wi-Fi link, a Bluetooth link, or a combination thereof, that is established between the respective devices 202, 204, 206 or 207 of system 200. The data storage provided by the cloud-based services 211 may store anonymized data, such as blood glucose measurement values, age, insulin sensitivity statistics based on gender, age, body mass index, and weight, or other forms of data. In addition, the cloud-based services 211 may process the anonymized data from multiple users to provide generalized information related to the various parameters used by the AP application. The cloud-based services 211 may also provide processing services for the system 200, such as performing the process 100 in the example of FIG. 1 or additional processes, such as that described with reference to FIGS. 3-6.

In an example, the device 202 includes a communication device 264, which as described above may be a receiver, a transmitter, or a transceiver that operates according to one or more radio-frequency protocols, such as Bluetooth, Wi-Fi, a near-field communication standard, a cellular standard, that may enable the respective device to communicate with the cloud-based services 211. For example, outputs from the sensor 204 or the medical device (pump) 202 may be transmitted to the cloud-based services 211 for storage or processing via the transceivers of communication device 264. Similarly, medical device 202, management device 206 and sensor 204 may be operable to communicate with the cloud-based services 211 via the communication link 288.

In an example, the respective receiver or transceiver of each respective device, 202, 206 or 207, may be operable to receive signals containing respective blood glucose measurement values of the number of blood glucose measurement values that may be transmitted by the sensor 204. The respective processor of each respective device 202, 206 or 207 may be operable to store each of the respective blood glucose measurement values in a respective memory, such as 223, 263 or 273. The respective blood glucose measurement values may be stored as data related to the AP application, such as 229, 249, 269 or 279. In a further example, the AP application operating on any of the management device 206, the smart accessory device 207, or sensor 204 may be operable to transmit, via a transceiver implemented by a respective communication device, 264, 274, 246, a control signal for receipt by the medical device 202. In the example, the control signal may indicate an amount of insulin included, for example, in the reduced-constraint meal bolus or the like, to be expelled by the medical device 202.

Various operational scenarios and examples of processes performed by the system 200 are described herein. For example, the system 200 may be operable to implement the process example of FIG. 1. In addition, the system may be operable to implement other operational examples that account for consumption of a meal by determining a reduced-constraint meal correction bolus.

In the operational example, the processor 261 of the personal diabetes management device 206 may be operable to use the number of blood glucose measurement values to determine the follow-up reduced-constraint meal bolus dosage. For example, the processor 261 may determine a sum of the blood glucose measurement values obtained within a predetermined period of time. It may be helpful to explain the calculation of a sum of the blood glucose measurement values obtained over a predetermined period of time, with reference to FIG. 5. FIG. 5 illustrates the receipt of blood glucose measurement values over a period of time and the summation of blood glucose measurement values over the period of time. In the example of FIG. 5, the blood glucose measurements received over a half-hour of time may be 140 mg/dL, 145 mg/dL, 155 mg/dL, 155 mg/dL, 165 mg/dL, and 175 mg/dL. The processor 261 may compare the sum (i.e., 140+145+155+158+165+175=938 mg/dL) to an aggregated blood glucose measurement value threshold value (e.g., 925 mg/dL), and in response to a result of the comparison indicating the sum has exceeded the aggregated blood glucose measurement threshold value (i.e., 938>925), the processor may determine the follow-up reduced-constraint meal bolus dosage. Conversely, in situations when the result of the comparison indicates the sum is below the aggregated blood glucose measurement threshold value, the processor may not implement the determination of the follow-up reduced-constraint meal bolus dosage and may wait for additional evidence of consumption of a meal. The additional evidence may be, for example, additional blood glucose measurement values that are used in a later calculation of the sum of the additional blood glucose measurement values and a comparison to an aggregated blood glucose measurement threshold value, or an input to a graphical user interface, such as 268, indicating a meal announcement.

In another example, the processor 261 may receive an input to a graphical user interface, such as 268, indicating a meal announcement and consumption of a meal. The input may be considered a first indication of consumption of the meal. The processor may determine whether the values of the received blood glucose measurement values are trending higher since receiving the indication of consumption of a meal. The processor 261, in response to a determination that the blood glucose measurement values are trending higher, may confirm consumption of the meal and generate another indication confirming consumption of the meal based on confirmation of consumption of the meal.

The processor 261 after receiving the confirmation may perform further functions, such as deriving a follow-up meal bolus dosage or the like.

It may be helpful to describe an operational example with reference to both FIG. 2 and the process flowchart of FIG. 3. FIG. 3 illustrates a flow chart of an example process for reducing meal correction safety constraints and delivering meal correction bolus dosages determined based on indications of meal consumption to compensate for rises in blood glucose measurement values. The process 300 combines the benefits of minimal user interaction with the system at each meal with automatic meal compensation through meal detection. In an example, there may be two use cases: 1) user indicates consumption, or imminent consumption, of a meal and 2) the user makes no announcement.

In the process 300, an indication of consumption of a meal may be received at 310. The indication may be, for example, an input into a user interface or an output from a meal detection algorithm. In this example, the processor of the personal diabetes management device when executing the artificial pancreas application may be operable to receive the indication of consumption of a meal. The received indication may be received from a meal detection and response application that is included in the programming code 267 or may be received in response to an input to user interface 268 (e.g., via a touch to a touchscreen).

At 320, the AP application may reduce meal detection thresholds for a meal detection and response algorithm. This first level may be a reduction of 25-35% of the meal detection thresholds. It is noted that meal detection thresholds are settings for triggering an indication of a meal from the meal detection and response algorithm, which are different from the meal correction bolus safety constraints. The meal correction bolus safety constraints are settings related to the timing and an amount of insulin that is to be administered to a user as part of a meal correction bolus dosage

For example, in use case 1, the sensitivity of the meal detection and response algorithm may be increased by reducing the threshold settings, and, as a result, detection of consumption of a meal to confirm the user's announcement of a meal may occur earlier and processing of a follow-up meal correction bolus dosage and delivery may be performed earlier.

In use case 2, the meal detection and response algorithm may be running in the background with the meal detection threshold settings at the initial settings for the meal detection and response algorithm (i.e., a meal announcement indication is more difficult to trigger) and has the higher thresholds of the initial settings. A rapid rise in blood glucose measurement values triggers the meal detection. In response, the AP application may cause the delivery of insulin later than in use case 1. If user fails to make an input that results in the meal announcement, the meal detection and response algorithm may continue to function with the higher initial thresholds. In such an example, it is anticipated that the meal detection and response algorithm may be more conservative.

Alternatively, if the user has pre-announced that a meal has been consumed or is about to be consumed (e.g., within the next 20 minutes or the like) by interacting with meal announcement button on a user interface of a PDM or smartphone, the AP application of the AID system may bypass the meal detection threshold requirements, and may initiate modification of the safety constraints for a meal-related bolus. Since there is uncertainty with the amount of carbohydrates in the meal, there is also a delay in delivering an entire reduced-constraint meal bolus. Instead of delivering the entire reduced-constraint meal bolus immediately in response to the user's announcement of a meal, a partial reduced-constraint meal bolus may be delivered. A partial reduced-constraint bolus may be, for example, 20% of the reduced-constraint meal bolus.

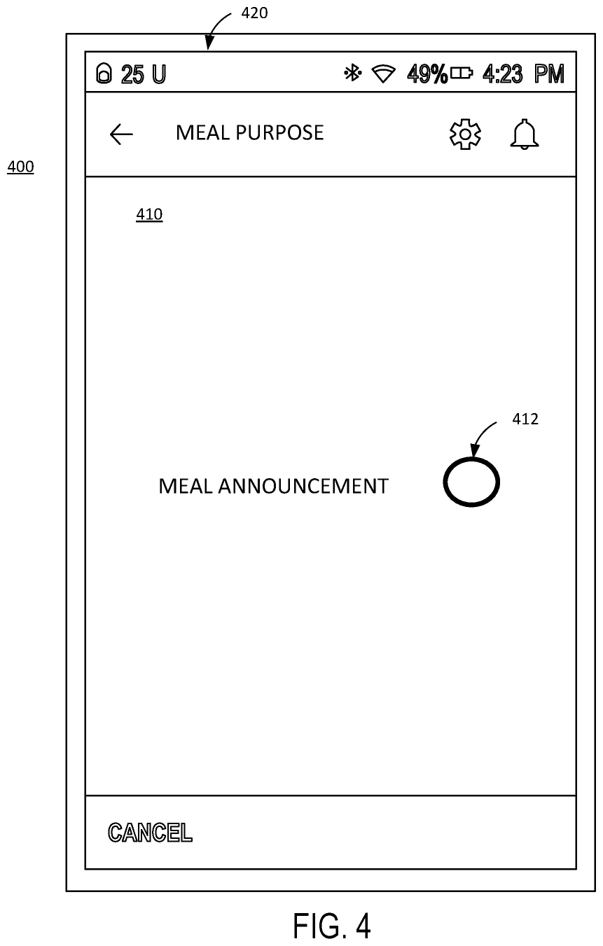

In the example of use case 2, the AP application may prompt a dialog box or other input, such as the meal announcement button (described with reference to FIG. 4 below) to be presented on a user interface of a personal diabetes management device or the like as a form of inquiry as to whether the user consumed a meal.

In 330 of FIG. 3, the processor may account for an amount of insulin-on-board the user when determining an amount of insulin to be delivered as a partial reduced-constraint meal bolus dosage. The partial reduced-constraint meal bolus dosage may be determined using meal bolus safety constraints that have been reduced to a first level based on the indication received at 310. For example, the AP application executing on the processor in response to the indication of consumption of the meal may decrease the post-prandial safety constraints from initial settings to a first level of reduced safety constraints. The AP application may calculate a reduced-constraint meal bolus dosage using the first level of reduced safety constraints. For example, the first level of reduced safety constraints may permit the AP application to administer a partial reduced-constraint meal bolus dosage of 50% of the estimated meal bolus requirements, instead of 10% of the estimated meal bolus requirements. Alternatively, the quantity of an immediate meal bolus dosage may not be modified and remain at 10% of the estimated meal bolus requirements, but the safety constraints for automated insulin delivery following this activation may be reduced. For instance, the one-time delivery constraint may be relaxed to allow delivery of up to 6 or more times the user's basal insulin needs, rather than 4 times the basal insulin needs. Further, the total insulin delivery over any period of 3 hours may be relaxed from no more than 9 times the user's basal needs, to 12 times the user's basal needs. Additionally, the insulin-on-board constraints may be reduced to allow the impact of previous insulin delivery to decay over a shorter duration of an insulin action curve—such as 2 hours, instead of a typical 3 hour curve of duration of insulin action.

The AP application executing on the processor may actuate delivery of the partial reduced-constraint meal bolus dosage (340). The partial reduced-constraint meal bolus dosage may be administered to begin compensating for a rise in blood glucose measurement values due to the consumption of the meal. In more detail, the processor, in response to the AP application calculating a partial reduced-constraint meal bolus dosage, may generate an actuation signal to be sent to a drug delivery device for actuation of a pump mechanism to deliver insulin according to the calculated reduced-constraint meal bolus dosage. The processor may be further operable to send an actuation signal (also referred to as a “control signal” or a “command signal”) to a drug delivery device, such as 202. In this example, the drug delivery device 202 includes a pump mechanism 224 and a reservoir 225. The reservoir 225 may be configured to contain insulin and is fluidly coupled to the pump mechanism 224. The pump mechanism 224 may be removably coupled to the user and is communicatively coupled to the processor 261 of the personal diabetes management device 206 and is operable to receive an actuation signal (control signal or command signal) and actuate in response to the received control or command signals. The pump mechanism 224 may expel, in response to the actuation signal from the processor, insulin from the reservoir according to the calculated reduced-constraint meal bolus dosage.

A concern with the rise in blood glucose measurement value is whether it is due to a random fluctuation due to a physiological change (e.g., sensitivity change) or a system event, or ingestion of a meal or a snack, and this creates uncertainty with how the AP application should respond. To mitigate the risks associated with the random fluctuation, the system is configured to wait a period of time to confirm whether the rise in blood glucose measurement value is sustained and remains consistent in the trend (e.g., about 2 (mg/dL)/minute). If the rise in blood glucose measurement value is sustained and remains consistent in the trend, the AP application of the AID system may determine that a meal has been consumed and initiate modification of the safety constraints for a meal-related bolus.

In the process 300, the processor may be operable to receive another indication confirming consumption of the meal 350. In contrast to the indication received at 310, the other indication may be from a different source. For example, if the indication at 310 was an input into a user interface, such as, a touch of a button on a graphical user interface the other indication may be an output from a meal detection algorithm. Conversely, if the indication at 310 was an output from a meal detection algorithm, the other indication may be in response to a user input into a user interface.

In response to the other indication confirming consumption of the meal at 350, the process 300 may reduce the meal safety constraints from the first level to a second level (355). For example, the initial safety constraints may only permit a meal correction bolus of 10 units (U) of insulin to be administered and the first level of safety constraints may permit 20 U of insulin to be delivered. Meanwhile, the second level of safety constraints may permit 30 U of insulin to be delivered. This enables the AP application to more aggressively compensate for a consistent increase in blood glucose measurement values and more quickly return the user's blood glucose measurement value to within range (such as within 0%-2%) of the user's target blood glucose value.

As an alternative to having two indications of a meal, another example process may modify the meal detection thresholds more aggressively than the previously described example In the example of the process 300 at 320, the reduction of the threshold for the meal detection and response algorithm may be between, for example, 50%-75%, 30%-60%, or the like, from the original meal detection threshold. The threshold may be set based on a blood glucose measurement value rise, for example, approximately 2 (mg/dL)/minute, approximately 3.5 (mg/dL)/minute, or the like.

With the meal bolus safety constraints reduced from the first level to a second level, the AP application may determine a follow-up meal bolus dosage (360). The determination of the follow-up reduced-constraint meal bolus dosage at 360 may further include steps, described below with reference to FIG. 5. For example, the AP application may sum the amount of blood glucose measurement values obtained over a predetermined period of time to obtain a total amount of blood glucose measurement values. The sum may be compared to an aggregated blood glucose measurement threshold value. In response to a result of the comparison indicating the sum has exceeded the aggregated blood glucose measurement threshold value, the follow-up reduced-constraint meal bolus dosage may be determined using the second level of meal bolus safety constraints. Alternatively, a curve fitting function may use the blood glucose measurement values over a predetermined period of time to determine a curve. The AP application may process the curve function to determine a total area under the curve. The total area may be compared to another aggregated blood glucose measurement threshold value that, in this example, is a threshold value related to the total area under the curve.

The AP application may cause the actuation of a drug delivery device to deliver insulin according to the follow-up meal bolus dosage (370) by sending an actuation signal to the drug delivery device to cause actuation of a pump mechanism. The actuation signal may include commands operable to cause delivery of insulin as well as an amount of insulin to be delivered that is equal to the amount in the follow-up meal bolus dosage. The delivery of insulin may be according to the follow-up meal bolus dosage. For example, with reference to FIG. 2, the processor 221 may generate a control signal that is applied to the pump mechanism 224 to expel an amount of insulin that is equal to the follow-up meal bolus dosage.

The meal detection-manual announcement operation (i.e., the receiving of an indication or another indication of consumption of a meal) may be captured, for example as three scenarios:

1) User announcement followed by meal. In this case, a user inputs via a user interface a meal announcement, such as touching a button or the like. The AP application is anticipating the user's blood glucose to rise. If the system detects a meal, then it validates the user input. The size of the meal bolus, size of meal ingestion, and how much duration of time to wait to deliver the bolus, and what proportion of the bolus to be delivered, can all be identified using past history of meals and the resulting changes in blood glucose measurement values, measured blood glucose values after the announcement and any other user inputs related to the announced meal.

2) User announcement but missed meal. In this type of situation, the user perhaps may have inadvertently pressed the announcement button or is delayed in consuming the meal (e.g., the user was going to eat alone but then decides to wait for company). The meal detection and response algorithm may determine 20-40 minutes after the user announcement that consumption of a meal cannot be detected based on a rise in blood glucose measurement values. If a blood glucose measurement value rise is not detected, the AP application may generate a prompt to interact with the user and confirm that a meal has been ingested. For example, the AP application may generate an audible notification for output from a speaker of the user interface with the presentation of the meal announcement button or meal announcement confirmation. In an example, a timer may be set when a user interacts with the meal announcement button, at which point the safety constraints are reduced in response to the meal announcement. If after an hour elapses since the user interaction with the meal announcement button and the meal detection algorithm does not detect a meal, the AP application may reset the safety constraints from the meal announcement safety constraints to the prior safety constraints. In either example, the AP application may alert the user, ensure that an excessive bolus is not given, or both.

3) Unannounced meal. In this case, the AP application via the meal detection and response algorithm may be operable to detect the meal, administer an initial bolus, perhaps estimate meal size, and administer a follow-up meal bolus upon confirmation of consumption of a meal (e.g., continued upward trending of the user's blood glucose measurement values).

Some of the foregoing examples referenced a graphical user interface presented on the user interface 268 of the personal diabetes management device 206. FIG. 4 illustrates an example of a user interface usable with the examples described in FIGS. 1-3. In the examples of FIGS. 1 and 3, the process steps of 120 of FIGS. 1 and 310 and 350 of FIG. 3, respectively, the user may interact with the user interface that includes a one-touch meal announcement button (shown in the example of FIG. 4). For reducing the burden on the use and to prevent the AID system from being a nuisance to the user, no additional prompts may be included related to meal size, number of calories, meal composition, or the like. For example, FIG. 4 shows an example of a graphical user interface 420 presented on a touchscreen 410 of a portable computing device 400. The graphical user interface 420 may, for example, be presented on a touchscreen device.

For example, with reference to FIGS. 2 and 4, the graphical user interface 420 and the meal announcement button 412 may be presented on user interface 278 of the smart accessory device 207, the user interface 268 of the management device 206 of FIG. 2.

The design of the meal announcement button 412 may be coupled with robust meal detection as explained with reference to the examples of FIGS. 1-3 above that allows the AP application to address at least two key issues of: 1) keeping the graphical user interface 420 simple, and 2) providing good glycemic control. Note that additional input mechanisms and methodologies may be used. For example, as shown the graphical user interface may receive a direct input from the user to a specific region of the graphical user interface (i.e., the meal announcement button 412), an input to a designated input device (e.g., a button (not shown) on the portable computing device), a voice input, a gesture input (hand or facial) to a camera. The indication or an announcement of a meal indicates consumption of a meal or indication of an intention to ingesting a meal by the user, or a rise in blood glucose measurement values that are indicative of a meal being consumed or having been consumed.

In an example, after a user announces a meal by touching the meal announcement button 412, the AP application may implement a process in which a large meal correction bolus is not triggered for delivery immediately, but instead the AP application may lower the meal detection threshold of the meal detection and response algorithm by a significant amount, such as 50-70%, mentioned above in order to increase the prior probability that a meal has been consumed. As such, the meal detection and response algorithm may be operable to analyze blood glucose measurement values in an attempt to estimate a size of the meal and trigger an appropriate meal correction bolus. In this case, the AP application may determine the appropriate meal correction bolus based on meal correction bolus safety constraint settings made in response to the meal announcement input.

The presence of the meal announcement button 412 is advantageous because it reduces the amount of time required for the user to indicate to the AP application that a meal is being consumed, about to be consumed, or has been consumed. In addition, by being only a single button, the user does not have to evaluate their meal for nutritional composition, a purpose of the meal or size of the meal, which in turn reduces the risk to the AP application in determining an incorrect dose of insulin and an inaccurate schedule for delivering the dose of insulin, among other risks due to incorrect information provided by the user.

The techniques described herein for providing safety constraints for a drug delivery system (e.g., the system 200 or any component thereof) may be implemented in hardware, software, or any combination thereof. For example, the system 200 or any component thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In the example of FIG. 5, the graphic illustrates analysis of blood glucose measurement values received from a blood glucose sensor according to the examples described with respect to FIGS. 1 and 3. The graph 500 shows a chart with the X axis as continuous glucose monitor (CGM) sensor output in units of mg/dL and the Y axis as time in minutes since a user interaction with a meal button. The graph 500 shows the blood glucose measurement values from the sensor 530 as black circles at different mg/dL values and the target blood glucose value setting as 110 mg/dL. A number of blood glucose measurement values are shown in the graph over a period of time that spans approximately 80 minutes. A blood glucose measurement value from the CGM may be provided to the AP application approximately every 5 minutes. The striped area beneath the curve 510 is the area under the curve 501 and is an aggregate of the respective blood glucose measurement values received for approximately 60 minutes after the user announced a meal by interacting with a meal announcement button, such as 412 in FIG. 4, at A. As mentioned above, the aggregate of the respective blood glucose measurement values may be compared to a threshold ((mg/dL)*minute). In the example of FIG. 4, the threshold may be 3500 (mg/dL)*minute and the total area under the curve 501 may be equal to approximately 3650 (mg/dL)*minute.

While the example of FIG. 5 shows the time at B since user interaction with the meal button being 60 minutes for determining the area under the curve 501, other time frames may be used such as 30 minutes, 45 minutes or 90 minutes.

In the example, when the user interacts with the meal button at A, the AP application may be operable to set time equal to zero or begin a counter that increments at particular times or the like. In addition, the AP application may reduce the safety constraints related to administration of a meal correction bolus in response to the user activation of the meal button in the graphical user interface, and cause delivery of the reduced meal correction bolus. The reduced meal correction bolus may be determined based on the formula:

$$\frac{( \begin{matrix}( \begin{matrix}blood glucose measurement value around the time \\ when the user interacts with the meal button at A\end{matrix} ) - \\ ( user ' s target blood glucose value setting )\end{matrix} ) }{( modified correction factor - Insulin \text{-} on \text{-} board}$$

Note that the sensor provides a blood glucose measurement value at approximately 5 minute intervals and there is no way to account for when a user may interact with the meal announcement button so the blood glucose measurement value that is relevant to time zero is the blood glucose measurement value received immediately preceding the input at A regardless of whether the immediately preceding blood glucose measurement value was received 1 second or 4 minutes and 59 seconds prior to the input at A. Therefore, “around,” in the equation above, may mean, in an example, the blood glucose measurement value provided within approximately 5 minutes before or after the user interaction with the meal button at A. Alternatively, “around” may mean, in other examples, the blood glucose measurement value provided within approximately 2 minutes, 10 minutes, 15 minutes, or the like before or after the user interaction with the meal button at A. The receipt of the blood glucose measurement value is shown as occurring exactly at the time of the user activation of the meal announcement button for ease of illustration. In the example of FIG. 5, the blood glucose measurement value is 143 mg/dL and the user's target blood glucose value setting is 110 mg/dL. The correction factor may range from a ratio of 1:20 (1 U of insulin drops user's glucose by 20 mg/dL) to 1:200 (1 U insulin drops user's glucose by 200 mg/dL), or the like. The modification to the correction factor is not shown but may be approximately 0.8 or the like, and the amount of insulin-on-board may change based on a time of day, an activity of the user (e.g., exercising, manual labor, or the like), a time since last meal, and the like.

In the example, the AP application is configured by programming code to wait in response to an indication from the input to the meal announcement button for another indication from the meal detection and for a response from the user that confirms that the user has consumed a meal. In the example of FIG. 5, the confirmation is by way of detecting an aggregated amount of blood glucose measurement values over a predetermined period of time. The predetermined period of time is the amount of time the AP application is configured to wait. In the graph 500, the predetermined period of time at B is 60 minutes (i.e., the amount of time from when the user interacts with the meal button at A). Of course, other predetermined periods of time may be used based on a user's physiology. For example, the user may process insulin more quickly than another user so the AP application may determine that a shorter predetermined period of time (e.g., 45 minutes) may be appropriate than the 60 minutes shown in the graph 500.

The AP application may be operable to process the received blood glucose measurement values by applying a curve fitting function to the blood glucose measurement values received within the predetermined period of time. The AP application may be operable to determine the area under the curve during the period of time (i.e., 60 minutes). Note that more precise curve fitting functions may be used and the straight line 510 is used for ease of illustration and explanation. From the graph 500, the area under the curve 501 is equal to the total area under a curve formed using the blood glucose measurement values 530 obtained within a predetermined period of time (i.e., 60 minutes). As mentioned with reference to other examples, the AP application may be operable to compare the total area, in this example, 3650 (mg/dL)*minute to an aggregated blood glucose measurement threshold value (e.g., 3500 (mg/dL)*minute). In an example, the area under the curve 510 may be determined using a definite integral of the curve 510 over the predetermined period of time. Of course, the predetermined period of time may be different than 60 minutes, such as 30 minutes, 45 minutes or 90 minutes. The AP application in response to a result of the comparison indicating the total area has exceeded the aggregated blood glucose measurement threshold value (i.e., 3650>3500) may determine a follow-up reduced-constraint meal bolus dosage because exceeding the aggregated blood glucose measurement threshold value is an indication that a meal has been consumed and the meal detection and response algorithm confirms that the meal announcement is correct. The follow-up reduced-constraint meal bolus may be determined based on a different formula (modified based on a further reduction of safety constraints):

$$\frac{B G M V - T a r g e t B G V}{C F} ,$$

where BGMV equals blood glucose measurement value, BGV equals blood glucose value and CF means correction factor.

In this follow-up reduced-constraint meal bolus, the insulin-on-board is not subtracted out so the reduction blood glucose measurement value back to target blood glucose value setting is more aggressively pursued.

In an operational example, a processor executing the meal detection and response algorithm may determine a trend of blood glucose measurement values, such as 530, based on a number of blood glucose measurement values received within a first time range before (e.g., from time 0 to time (−) 10) and a second time range after receipt of the indication of consumption of a meal (e.g., time 0 to time 60). The blood glucose measurement value trend may be an increase in blood glucose measurement values that is a consistent increase over a predetermined number of blood glucose measurement values, such as the second time range after receipt of the indication or another such as time 0 to time 30, or the like. The processor may compare the determined trend to a meal detection blood glucose trend threshold. In response to the trend meeting or exceeding the meal detection blood glucose trend threshold, the processor may decrease the post-prandial safety constraints further from a first level of reduced safety constraints to a second level of reduced post-prandial safety constraints. A follow-up meal bolus dosage may be calculated using the second level of reduced safety constraints and a signal may be output actuating delivery of insulin according to the calculated follow-up meal bolus dosage.

FIG. 6 is a flow chart of an example of a process for generating and using a machine-learning model according to some aspects of the disclosed examples. Machine learning is a branch of artificial intelligence that relates to mathematical models that can learn from, categorize, and make predictions about data. Such mathematical models, which can be referred to as machine-learning models, can classify input data among two or more classes; cluster input data among two or more groups; predict a result based on input data; identify patterns or trends in input data; identify a distribution of input data in a space; or any combination of these. Examples of machine-learning models can include (i) neural networks; (ii) decision trees, such as classification trees and regression trees; (iii) classifiers, such as Bayes classifiers, logistic regression classifiers, ridge regression classifiers, random forest classifiers, least absolute shrinkage and selector (LASSO) classifiers, and support vector machines; (iv) clusterers, such as k-means clusterers, mean-shift clusterers, and spectral clusterers; (v) factorizers, such as factorization machines, principal component analyzers and kernel principal component analyzers; and (vi) ensembles or other combinations of machine-learning models. In some examples, neural networks can include deep neural networks, feed-forward neural networks, recurrent neural networks, convolutional neural networks, radial basis function (RBF) neural networks, echo state neural networks, long short-term memory neural networks, bi-directional recurrent neural networks, gated neural networks, hierarchical recurrent neural networks, stochastic neural networks, modular neural networks, spiking neural networks, dynamic neural networks, cascading neural networks, neuro-fuzzy neural networks, or any combination of these.

Different machine-learning models may be used interchangeably to perform a task. Examples of tasks that can be performed at least partially using machine-learning models include various types of scoring; bioinformatics; cheminformatics; and the like.

For example, machine learning may be used in the meal detection. The machine learning algorithm may be trained using blood glucose measurement value data extracted from a number of users. The training may enable the machine learning algorithm to identify a pattern, such as during a meal an average rise of blood glucose measurement value is 20 mg/dL over 10 minutes and consistently for an additional 10-20 minute interval. Other patterns may also be recognized as indicative of a meal, such as a blood glucose measurement value that remains flat at a high value above the target glucose, such as 50 mg/dL, despite recent delivery of a significant amount of insulin, such as 10 U. Further, a change in the rate of the user's glucose concentrations, even if it is decreasing, may indicate a meal ingestion, such as if the glucose concentration was dropping by 20 mg/dL per 5 minutes, but then started dropping by only 2 mg/dL per 5 minutes. The meal detection classifier may be trained so different rise characteristics may be categorized. The meal detection classifier may be generalized for mass usage by being trained using general blood glucose related data at first but may be further trained to be specific to the particular user based on further training using data of the user (either historical data, real-time data or a combination of both).

Machine-learning models can be constructed through an at least partially automated (e.g., with little or no human involvement) process called training. During training, input data can be iteratively supplied to a machine-learning model to enable the machine-learning model to identify patterns related to the input data or to identify relationships between the input data and output data. With training, the machine-learning model can be transformed from an untrained state to a trained state. Input data, such as a user's blood glucose measurement values or a number of different users' blood glucose measurement values, can be split into one or more training sets and one or more validation sets, and the training process may be repeated multiple times. The splitting may follow a k-fold cross-validation rule, a leave-one-out-rule, a leave-p-out rule, or a holdout rule. An overview of training and using a machine-learning model is described below with respect to FIG. 6, which is a flowchart of an example of a process for training and using a machine-learning model according to some aspects of the foregoing examples.

The process 600 includes several steps, for example, in block 604, training data is received. In some examples, the training data is received from a remote database or a local database that is configured to store blood glucose measurement values, time stamps related to the respective blood glucose measurement values, meal detection indications from both a meal detection response algorithm and a user interaction with the meal announcement button and the like. The training data may further be constructed from various subsets of data, or input by a user. The training data can be used in its raw form for training a machine-learning model or pre-processed into another form, which can then be used for training the machine-learning model. The training data may be processed by a cloud-based service and used to populate a model used by the meal detection and response algorithm or by the AP application when setting the safety constraints, or the like. For example, the raw form of the training data can be smoothed, truncated, aggregated, clustered, or otherwise manipulated into another form, which can then be used for training the machine-learning model. In examples, the training data may include anonymized user information, blood glucose measurement information, insulin delivery history information and/or the like.

In block 606, a machine-learning model is trained using the training data. The machine-learning model can be trained in a supervised, unsupervised, or semi-supervised manner. In supervised training, each input in the training data is correlated to a desired output. This desired output may be a scalar, a vector, or a different type of data structure such as text or an image. This may enable the machine-learning model to learn a mapping between the inputs and desired outputs. In unsupervised training, the training data includes inputs, but not desired outputs, so that the machine-learning model must find structure in the inputs on its own. In semi-supervised training, only some of the inputs in the training data are correlated to desired outputs.

In block 608, the machine-learning model is evaluated. For example, an evaluation dataset can be obtained, for example, via user input or from a database. The evaluation dataset can include inputs correlated to desired outputs, such as a range for a target blood glucose measurement value. The inputs can be provided to the machine-learning model and the outputs from the machine-learning model can be compared to the desired outputs. If the outputs from the machine-learning model closely correspond with the desired outputs, the machine-learning model may have a high degree of accuracy. For example, if 90% or more of the outputs from the machine-learning model are the same as the desired outputs in the evaluation dataset, e.g., a target blood glucose value or the like, the machine-learning model may have a high degree of accuracy. Otherwise, the machine-learning model may have a low degree of accuracy. The 90% number is an example only and the accuracy of which is dependent on the problem being solved, which in this case is compliance with post-prandial safety constraints and the data, such as target blood glucose measurement value, blood glucose measurement values, and the like.

In some examples, if the machine-learning model has an inadequate degree of accuracy for a task, the process can return to block 606, where the machine-learning model can be further trained using additional training data or otherwise modified to improve accuracy. If the machine-learning model has an adequate degree of accuracy for the task, the process can continue to block 610.

In block 610, new data is received. In some examples, the new data is received from a remote database or a local database, constructed from various subsets of data, or input by a user. The new data may be unknown to the machine-learning model. For example, the machine-learning model may not have previously processed or analyzed the new data. In some examples, the new may be provided to the process at 604 for use as training data.

In block 612, the trained machine-learning model is used to analyze the new data, such as real-time receipt of a blood glucose measurement value from a glucose sensor and provide a result (e.g., the user's blood glucose measurement values are trending higher at a rate of 3 (mg/dL)/minute or the like). For example, the new data can be provided as input to the trained machine-learning model. The trained machine-learning model can analyze the new data and provide a result that includes a classification of the new data into a particular class (e.g., meal detected or meal NOT detected), a clustering of the new data into a particular group (e.g., meal detected or meal NOT detected), a prediction based on the new data (e.g., blood glucose measurement value will reduce to within a range (e.g., 0-2%, 5%-10% or the like) of a target blood glucose value setting in 30 minutes, or the like), or any combination of these.

In block 614, the result is post-processed. For example, the result can be added to, multiplied with, or otherwise combined with other data as part of a job. As another example, the result can be transformed from a first format, such as a time series format, into another format, such as a count series format. Any number and combination of operations can be performed on the result during post-processing.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A device, comprising:
a processor;
a memory storing programming code, an artificial pancreas application, and operable to store data related to the artificial pancreas application, wherein the programming code and the artificial pancreas application are executable by the processor;
a touchscreen operable to present a graphical user interface and receive inputs; and
a transceiver operable to receive and transmit signals containing information usable by or generated by the artificial pancreas application, wherein:
the artificial pancreas application includes post-prandial safety constraints related to administering of insulin in response to consumption of a meal, and
the processor when executing the artificial pancreas application is operable to:
receive an indication of consumption of the meal;
in response to the indication of consumption of the meal, decrease the post-prandial safety constraints from initial settings to a first level of reduced safety constraints;
calculate a reduced-constraint meal bolus dosage using the first level of reduced safety constraints; and
generate a signal to actuate delivery of insulin according to the calculated reduced-constraint meal bolus dosage.

2. The device of claim 1, wherein the artificial pancreas application includes programming code that when executed by the processor, causes the processor to be further operable to:
receive, via the transceiver, signals, wherein each signal contains a respective blood glucose measurement value of a number of blood glucose measurement values; and
store each respective blood glucose measurement value of the number of blood glucose measurement values in the memory.

3. The device of claim 1, wherein the processor when calculating follow-up meal bolus dosage is operable to:
sum a total amount of blood glucose measurement values obtained within a predetermined period of time;
compare the sum to an aggregated blood glucose measurement value threshold value; and
in response to a result of the comparison indicating the sum has exceeded the aggregated blood glucose measurement value threshold value, determining a follow-up reduced-constraint meal bolus dosage.

4. The device of claim 1, wherein the processor is further operable to:
received a number of blood glucose measurement values within a predetermined period of time;
determine that a user has ingested a meal based on an analysis of the obtained blood glucose measurement values with respect to a blood glucose measurement value threshold value; and
in response to the determination, generate the indication that a meal has been ingested.

5. The device of claim 1, wherein the processor when executing the artificial pancreas application is operable to:
after a period of time has elapsed since receipt of the indication of consumption of the meal, receive a notification that consumption of a meal is unconfirmed, wherein the indication was generated in response to a meal announcement received from a user interface;
cease calculation of a follow-up meal bolus dosage; and
reset the reduced safety constraints from the first level to the initial settings for the post-prandial safety constraints.

6. The device of claim 1, wherein the processor when calculating the meal bolus dosage is operable to:
determine a trend of blood glucose measurement values based on a number of blood glucose measurement values received within a first time range before and a second time range after receipt of the indication of consumption of a meal, wherein the blood glucose measurement value trend is an increase in blood glucose measurement values that is a consistent increase over a predetermined number of blood glucose measurement values;
compare the determined trend to a meal detection blood glucose trend threshold;
in response to the trend meeting or exceeding the meal detection blood glucose trend threshold, decrease the post-prandial safety constraints further from the first level of reduced safety constraints to a second level of reduced post-prandial safety constraints;
calculate a follow-up meal bolus dosage using the second level of reduced safety constraints; and
output a signal actuating delivery of insulin according to the calculated follow-up meal bolus dosage.

7. The device of claim 6, wherein the processor, when outputting the signal actuating delivery is further operable to:
transmit, via the transceiver, the generated signal as a control signal for receipt by a medical device, wherein the control signal indicates an amount of insulin included in the reduced-constraint meal bolus to be expelled by the medical device.

8. A non-transitory computer readable medium embodied with programming code executable by a processor, and the processor when executing the programming code is operable to:
maintain track of an amount of pre-existing insulin-on-board for a user;
receive an announcement of a meal, wherein the meal announcement is a signal in response to an indication of meal-related activity;
determine a reduced-constraint meal bolus dosage based on a target blood glucose value setting, the amount of pre-existing insulin-on-board for the user and revised safety constraints;
determine an amount of time that has elapsed since a last bolus was delivered;
compare the amount of elapsed time to a threshold; and
in response to a result of the comparison indicating the elapsed time is less than the threshold, generate a signal to actuate a pump mechanism to deliver the meal bolus dosage.

9. The non-transitory computer readable medium of claim 8, further embodied with programming code executable by the processor, and the processor when executing the programming code to determine the reduced-constraint meal bolus dosage is operable to:
obtain an amount of insulin-on-board for the user, wherein the amount of insulin-on-board is an estimate of the amount of insulin remaining in the user's body that continues to reduce the user's blood glucose measurement values;
obtain the reduced-constraint meal bolus dosage by modifying a recommended meal bolus dosage of insulin by subtracting the obtained amount of insulin-on-board from the recommended meal bolus dosage; and
output the reduced-constraint meal bolus dosage.

10. The non-transitory computer readable medium of claim 8, further embodied with programming code executable by the processor, and the processor when executing the programming code is operable to:
receive an indication from a meal detection algorithm that a user has ingested a meal; and
generate the announcement of the meal based on the received indication.

11. The non-transitory computer readable medium of claim 8, further embodied with programming code executable by the processor, and the processor when executing the programming code is operable to:
analyze a trend of previous blood glucose measurement values; and
in response to the analysis indicating that the trend of previous blood glucose measurement values is rising, generate the received indication and the announcement of the meal.

12. The non-transitory computer readable medium of claim 8, further embodied with programming code executable by the processor, and the processor, when executing the programming code to determine the reduced-constraint meal bolus dosage, is operable to:
obtain a total daily insulin of the user;
obtain a user's blood glucose measurement values over a period of time; and
utilize the obtained user's total daily insulin and the obtained blood glucose measurement values in the determination of the reduced-constraint meal bolus dosage.

13. The non-transitory computer readable medium of claim 12, further embodied with programming code executable by the processor, and the processor, when executing the programming code to determine the reduced-constraint meal bolus dosage, is operable to:
determine a difference between the blood glucose measurement value of the user at a time when the meal announcement was received and the target blood glucose value setting;
divide the difference by a correction factor related to a user's insulin sensitivity to provide a proposed meal bolus dosage;
determine an amount of insulin-on-board for the user;
subtract the determined amount of insulin-on-board for the user from the proposed meal bolus dosage; and
output a result of the subtraction as the reduced-constraint meal bolus dosage.

14. The non-transitory computer readable medium of claim 8, further embodied with programming code executable by the processor, and the processor, when executing the programming code to determine the reduced-constraint meal bolus dosage, is operable to:
receive the announcement from a meal detection algorithm in response to an input from the user, an input to a designated input device, a voice input, a gesture input to a camera, an input to a touchscreen or a touchpad, or receive a determination of the meal-related activity from a meal detection algorithm in response to:
ingestion of a meal or an indication of an intention to ingesting a meal input by the user, or a rise in blood glucose measurement values that are indicative, based on a meal detection algorithm, of a meal being consumed or having been consumed.

15. A system, comprising:
a personal diabetes management device including:
a processor;
a memory storing programming code, an artificial pancreas application, and post-prandial safety constraints, and data related to the artificial pancreas application, wherein the programming code and the artificial pancreas application are executable by the processor; and
a transceiver operable to receive and transmit signals containing information of the artificial pancreas application; and
a drug delivery device, the drug delivery device including:
a pump mechanism operable to be removably coupled to a user; and
a reservoir operable to contain insulin and is fluidly coupled to the pump mechanism,
wherein the processor of the personal diabetes management device when executing the artificial pancreas application is operable to:
receive an indication of consumption of a meal;
determine an amount of insulin to be delivered as a partial reduced-constraint meal bolus dosage using meal bolus safety constraints that are reduced to a first level based on the received indication;
actuate delivery of the partial reduced-constraint meal bolus dosage;
receive another indication confirming consumption of the meal;
determine a follow-up meal bolus dosage based on meal bolus safety constraints reduced to a second level from the first level; and
actuate delivery of insulin according to the follow-up meal bolus dosage.

16. The system of claim 15, wherein the processor of the personal diabetes management device, when determining the follow-up meal bolus dosage based on meal bolus safety constraints reduced to a second level from the first level, is operable to:
sum a total area under a curve of blood glucose measurement values obtained from a blood glucose sensor within a predetermined period of time;
compare the total area to an aggregated blood glucose measurement value threshold value; and
in response to a result of the comparison indicating the total area has exceeded the aggregated blood glucose measurement value threshold value, determine the follow-up reduced-constraint meal bolus dosage.

17. The system of claim 15, the processor of the personal diabetes management device, when receiving the indication of consumption of the meal or receiving the other indication confirming consumption of the meal, is operable to receive an input into a user interface or an output from a meal detection and response algorithm.

18. The system of claim 15, further comprising:
a blood glucose sensor communicatively coupled to the processor of the personal diabetes management device and operable to:
measure a blood glucose value at a predetermined time interval, wherein the blood glucose sensor provides a number of blood glucose measurement values.

19. The system of claim 18, wherein the processor of the personal diabetes management device is operable to:
receive, from the blood glucose sensor, a number of blood glucose measurement values obtained over a period of time;
determine whether the number of blood glucose measurement values are trending higher since receiving the indication of consumption of a meal;
in response to a determination that the number of blood glucose measurement values are trending higher, confirm consumption of the meal; and
generate the other indication confirming consumption of the meal based on confirmation of consumption of the meal.

20. The system of claim 15, wherein the personal diabetes management device further comprises:
a touchscreen operable to present a graphical user interface; and
wherein the processor of the personal diabetes management device when executing the artificial pancreas application is further operable to:
receive the indication of consumption of a meal via a meal announcement from a meal detection algorithm in response to:
an input from the user, an input to a designated input device, a voice input, a gesture input to a camera, or an input to a touchscreen or a touchpad, or
a determination of meal-related activity by the meal detection algorithm, wherein meal-related activity is ingestion of a meal and indication of an intention to ingesting a meal by the user, or a rise in blood glucose measurement values that are indicative of a meal being consumed or having been consumed.
